(19) United States
(12) Patent Application Publication
(10) Pub. No.: US 2020/0040307 A1
(43) Pub. Date: Feb. 6, 2020

(54) CANCER STEM CELL

(71) Applicant: CancerStem Tech Inc., Ibaraki-shi, Osaka (JP)

(72) Inventors: Xin Wu, Nishinomiya-shi, Hyogo (JP); Masaki Mori, Toyonaka-shi, Osaka (JP); Hirofumi Yamamoto, Osaka-shi, Osaka (JP)

(21) Appl. No.: 16/499,094

(22) PCT Filed: Mar. 30, 2018
(86) PCT No.: PCT/JP2018/013561

§ 371 Date: Sep. 27, 2019

(30) Foreign Application Priority Data
Mar. 30, 2017 (JP) 2017-066600

(51) Int. Cl. C12N 5/095; C12N 15/86; G01N 33/15; G01N 33/50

(52) U.S. Cl. CPC C12N 5/0695; G01N 33/5011; G01N 33/15; C12N 15/86

(57) ABSTRACT

The purpose of the present invention is to provide: a cancer stem cell having an excellent ability to form tumor tissue; and a method for establishing the cancer stem cell. Cells having a low metabolic function of 26S proteasome are isolated and concentrated from a cancer stem cell group containing cancer stem cells, thereby obtaining cancer stem cells which can form tumor tissue in a living body even when the number of cells is 200 or less.

### TECHNICAL FIELD

The present invention relates to a cancer stem cell having an excellent ability to form tumor tissue, and being capable of forming tumor tissue even with a small number of cells. The present invention also relates to a method for establishing the cancer stem cell.

### BACKGROUND ART

Cancer cells have a self-proliferation potency, and have the property of being capable of infiltration into a surrounding tissue and metastasis into a distant tissue. However, it has been found that not all cancer cells forming cancer tissue have such properties, but cancer cells developing or progressing cancer are cancer stem cells that rarely exist in cancer cells. Similar to normal stem cells, cancer stem cells exhibit an undifferentiated surface morphology, have a self-proliferation potency and differentiation potency, and have the property of producing any cancer cells constituting cancer tissue that are in various differentiation stages. In other words, cancer stem cells are considered to be the basis for generating a majority of cancer cells by differentiation, while maintaining the same cells as themselves by self-proliferation in cancer tissue.

For controlling cancer, it is important to control cancer stein cells by clarifying the properties of cancer stem cells. Accordingly, cancer stem cells are useful for drug discovery and as a tool for developing a diagnostic agent, so that establishment of their samples is desired. Conventionally, establishment of cancer stem cells has been performed using stem cell surface markers, side populations, ability to form spheres and the like as indicators. However, cancer stem cells rarely exist in cancer cells constituting cancer tissue, and also cancer stem cells established by conventional methods do not have high ability to form tumor tissue. Therefore, when the number of cells is low, tumor tissue cannot be formed in model animal. Accordingly, there is a disadvantage in that if a large number of cancer stem cells are not prepared, a tumor animal model cannot be created.

In the background of such a prior art, it is desired to develop a technique of manufacturing samples of cancer stem cells which can form tumor tissue even in a few number of cells and satisfy a medical industry.

### SUMMARY OF THE INVENTION

### Problems to be Solved by the Invention

It is a main object of the present invention is to provide a cancer stem cell having an excellent ability to form tumor tissue, and a method for establishing the cancer stem cell.

### Means for Solving the Problem

The present inventors have carried out earnest studies to solve the above problems, and found that cancer stem cells capable of forming tumor tissue even with as low as 200 or less cells in vivo are obtained by separating and enriching cells having a low metabolic function of 26s proteosome from a cancer stem cell group containing the cancer stem cells. Furthermore, the present inventors have found that cancer stem cells having an extremely superior ability to form tumor tissue are obtained by further separating cancer stem cells highly expressing CD44v9 from the enriched cells (cancer stem cells) having a. low metabolic function of 26s proteosome. The present invention has been completed by further conducting studies based on these findings.

In other words, the present invention provides an invention of the aspects described below.

Item 1. A cancer stem cell capable of forming tumor tissue with 50 or less cells.

Item 2. The cancer stem cell according to item 1, which is capable of forming tumor tissue with one cell.

Item 3. The cancer stem cell according to item 1 or 2, which highly expresses CD44v9.

Item 4. The cancer stem cell according to any one of items 1 to 3, which is Panc-1 3-4 CST 001 line (Accession number: NITE BP-02449).

Item 5. A method for separating a cancer stem cell, including a step of separating and enriching a cell having a low metabolic function of 26s proteosome from a cancer cell group containing the cancer stem cell.

Item 6. The method for separating a cancer stem cell according to item 5. wherein the step of separating and enriching a cell having a low metabolic function of 26s proteosome is conducted by transducing a labeled ornithine decarboxylase-degron into the cancer cell group.

Item 7. The method for separating a cancer stem cell according to item 6, including further separating a cell highly expressing CD44v9 from the cell having a low metabolic function of 26s proteosome, the cell being enriched.

Item 8. A method for screening a cancer therapeutic agent, including screening a cancer therapeutic agent using the cancer stem cell according to any one of items 1 to 4.

Item 9. A method for evaluating efficacy of a cancer therapeutic agent, including evaluating efficacy of a cancer therapeutic agent using the cancer stem cell according to any one of items 1 to 4.

### Advantages of the Invention

According to the cancer stem cells of the present invention, tumor tissue can be formed in model animal even with as low as 200 or less cells (in one particularly preferred aspect, one cell), and thus a tumor animal model can be more easily created. In addition, the cancer stem cells of the present invention can be used for screening of a cancer therapeutic agent, evaluation of efficacy of a cancer therapeutic agent and the like, and thus can contribute to improvement of cancer therapeutic technologies. In addition, by clearing the characteristics of the cancer stem cells of the present invention, it is possible to elucidate the mechanisms of development, progression, metastasis and the like of cancer. As a result, they can contribute to innovative drug discovery and development of therapies leading to curative treatment of cancer.

### BRIEF DESCRIPTION OF THE DRAWINGS

FIG. 1a shows the result of observation of a cancer stem cell line (about 0.06%) with a fluorescence microscope visualized by introducing an ornithine decarboxylase (ODC)-degron system into a pancreatic cancer cell line Panc-1. FIG. 1b shows the result of observation of a cancer stem cell line before and after enrichment of the cancer stem cell line. The upper views in FIG. 1b are the result of observation of a cell population before enrichment, and the lower views are the result of observation of a cell population after enrichment.

FIG. 2 shows the result of confirmation of the presence or absence of characteristics of cancer stem cells for a cancer stem cell population (ZsGreen+) and a non-cancer stem cell population (ZsGreen−, negative control). The result of evaluation of the ability to form spheres is shown in FIG. 2a, the result of evaluation of the anticancer drug resistance in FIG. 2b, the result of evaluation of the ability to express a stem cell marker in FIG. 2c, and the result of evaluation of the asymmetric division potency in FIG. 2d.

FIG. 3 is the result of observation of transplanted sites on mouse 6 weeks after 150 cells from a cancer stem cell population (ZsGreen+) and 150 cells from a non-cancer stem cell population (ZsGreen−) are each subcutaneously transplanted.

FIG. 4 shows the result of observation of transplanted sites on mouse 6 weeks after 150 cells from a cancer stem cell population highly expressing CD44v9 (ZsGreen−/CD44v9^{high}) and 150 cells from a non-cancer stem cell population highly expressing CD44v9 (ZsGreen−/CD44v9^{high}) are each subcutaneously transplanted.

FIG. 5 shows the result of observation of a transplanted site on mouse over time where only 1 cell from an established cancer stem cell line (super Panc-1 CSC) highly expressing CD44v9 has been subcutaneously transplanted.

### EMBODIMENTS OF THE INVENTION

1. Characteristics of Cancer Stem Cell etc.

The cancer stem cells of the present invention are characterized in that they can form tumor tissue with 200 or less cells. Conventionally established cancer stem cells cannot form tumor tissue in model animal with such a small number of cells, but the cancer stem cells of the present invention can form tumor tissue in model animal even with as low as 200 or less cells. Hereinafter, a detailed description is made of the cancer stem cells of the present invention.

In the present invention, cancer stem cells mean cells which can maintain an undifferentiated state, have a self-proliferation potency and differentiation potency, and produce cancer cells by differentiation.

One of the characteristics of the cancer stem cells of the present invention is that they can form tumor tissue with 200 or less cells. A suitable aspect of the cancer stem cells of the present invention can form tumor tissue with 150 or less, preferably 100 or less, 75 or less, 50 or less, 40 or less, or 30 or less, more preferably 1 to 20, further preferably 1 to 10, particularly preferably 1 to 5, 1 to 4, or 1 to 3 cells, most preferably 1 or 2 cells, or 1 cell.

In the present invention, it can be determined whether “tumor tissue can be formed in vivo” by confirming whether or not, after a predetermined number of cancer stem cells are administered to model animal, tumor tissue is formed at the administrated site. More simply, it can be determined by visually confirming whether or not, 6 weeks after a predetermined number of cancer stem cells are administered subcutaneously to a mouse, a tumor tissue having a diameter of 10 mm or more is formed at the administrated skin site.

In addition, a suitable aspect of the cancer stem cells of the present invention highly expresses CD44v9. CD44v9 is known as a cancer stem cell surface marker. By selecting cells having a high expression level of CD44v9 among the cancer stem cells, it is possible to provide cancer stem cells having remarkably excellent ability to form tumor tissue and being capable of forming tumor tissue in vivo even when the number of cells is 10 or less (in particular, one).

In the present invention, “highly expressing CD44v9” means that, when a population of cancer stem cells is subjected to FACS analysis using an anti-CD44v9 antibody labeled with a labeling substance such as a fluorescent substance, the detected amount of labeled substance(CD44v9 expression level) is observed 5 times or more, preferably 5 to 100 times, more preferably 10 to 100 times than the CD44v9 expression level of cells having a detected amount of labeled substance at the detection threshold. Here, the “detection threshold” refers to the detected amount of labeled substance observed when the labeled substance is detected without using the anti-CD44v9 antibody for the same stem cells (negative control). Note that the average expressed amount of CD44v9 on a normal cancer stem cell having a poor ability to form tumor tissue is less than 5 times than that on a cancer non-stem cell (a cancer cell that is not a stem cell) from the same separation source.

In addition, a preferred aspect of the cancer stem cells of the present invention (in particular, the cancer stem cells highly expressing CD44v9) has activated pathways involved in growth/progression of tumor tissue and/or metastasis/differentiation/proliferation of tumor cells. Specific indicators of such activated pathways include increased or decreased expression of at least one or more of genes related to the functions of the following (1) to (28): (1) growth of tumor, (2) cell movement of endothelial cell lines, (3) migration of endothelial cell line lines, (4) synthesis of carbohydrate, (5) synthesis of polysaccharide, (6) synthesis of protein, (7) synthesis of glycosaminoglycan, (8) concentration of lipid, (9) cell survival, (10) chemotaxis of cells, (11) cell viability, (12) permeability of vascular tissue, (13) chemotaxis, (14) release of eicosanoid, (15) metabolism of carbohydrate, (16) release of fatty acid, (17) synthesis of DNA, (18) migration of tumor cell lines, (19) differentiation of tumor cell lines, (20) proliferation of tumor cells, (21) tyrosine phosphorylation, (22) development of abdomen, (23) metastasis of cells, (24) cell movement, (25) movement of vascular endothelial cells, (26) progression of tumor, (27) organization of cytoskeleton and (28) activation of cells.

A preferred aspect of the cancer stem cells of the present invention may have increased or decreased expression of at least one or more of genes related to the above-mentioned functions (1) to (28). Among the functions (1) to (28), expression of genes related to preferably 5 or more, more preferably 10 or more, still more preferably 15 or more, particularly preferably 20 or more, still more preferably 25 or more, most preferably 29 (all) functions is increased or decreased. Here, as to “increased or decreased expression of genes”, in the case of a gene related to enhancement of the function, the expression needs to be increased, and in the case of a gene related to suppression of the function, the expression needs to be suppressed.

Specific genes involved in (1) growth of tumor include at least one selected from the group consisting of ADM, AFAPIL2, AKR1B 10, ALCAM, ANGPTL4, ATF3, BMP2, BMP4, CA9, CCDC88A, CCLS, CD82, CERS6, CLU, COL18A1, CST6, CTSB, CTSS, CTSV, CXCL8, DAPK1, DKK1, ERBB3, ERO1A, F3, FGF2, FGFR3, 1⁷N1, FST, GADD45A, GLDC, GRN, HDAC6, HRAS, ICAM2, IGFBP2, IL18, IL32, ITGB6, JAG1, KMT2A LGALS7/LGALS7B, LM02, LOX, LRIG1, LUM, MAOA, MCAM MIF, mir-25, MK167, MMPI, NOV, NR4A2, NRG1, NTS, PAKI. PFKFB3, PGF, PHILDA1, PLA2G4A, PPARGC1A, PPP2R2A, PTGS2, PIN, PTPN13, RORC, RRAD, S100A9, SEMA3B, SLC7A11, SNAI1, SNAI2, SOX2, SPARC, SRC, TP73, TR1M22, VCAN and VEGFC.

Specific genes involved in (2) cell movement of endothelial cell lines include at least one selected from the group consisting of ADM, ALCAM, ANGPTL4, COL18A1, CTSB, CX3CL1, CXCL8, FGF2, 1L18, NRG1, PLPP3, SRC and VEGFC.

Specific genes involved in (3) migration of endothelial cell lines include at least one selected from the group consisting of ALCAM, ANGPTL4, COL18A1, CTSB, CX3CL1, CXCL8, FGF2, 1L18NRG1, PLPP3, SRC and VEGFC.

Specific genes involved in (4) synthesis of carbohydrate include at least one selected from the group consisting of AGPAT1, ALDH1A1, APLN, BMP2, CCL5, CHST15, CSGALNACT1, CX3CL1, CXCL8, DKK1, DPAGT1, FGF2, FOXA2, GALNT5, GOT1, GRB10, GRK5, HAS3, HRAS, 1L18, LPL, MIF, NR4A2, NRG1, NTS, PP ARGC1A, PPP1CB, PPP1R3C, PTGER4, RGS2, SNCA, ST6GALN AC1, SYK, TIRAP and VEGFC.

Specific genes involved in (5) synthesis of polysaccharide include at least one selected from the group consisting of APIA, BMP2, CCL5, CSGALNACT1, CX3C1,1, DKK1, FGF2, FOXA2, GALNTS, GRB10, HAS3, HRAS, ILI 8, NRG1, PPARGC1A, PPPICB, PPP1R3C, PTGER4 and VEGFC.

Specific genes involved in (6) synthesis of protein include at least one selected from the group consisting of ADM, AFAP1L2, APLN, BMP2, BMP4, BMPRIB, C3, CBLB, CCDC88A, CCL5, CDKI, CHST15, CLN3, CNTN1, CSK, CX3CL1, CXCL8, DAB2, DAPK1. EDIL3, EPOR, FAA/120C, FGF2, FGFR3GRB10, GRK.5, HDAC6, HRAS, TOGAP3MAPK4, MIF, MMD, MMP1, NRG1, NTRK2, PAG1, PAK1, PDK1, PDK2, PELI2, PLPP3, PPARGC1A, PRR5L, PSEN2, PTGER4, PTN RORC SNCA., SRC,ST6GAL1, STK19, STK36, SYK, TF, UCN, VEGFC and ZBED3.

Specific genes involved in (7) synthesis of glycosaminoglycan include at least one selected from the group consisting of BIMP2, CCL5, CSGALNACT1, CX3CL1, DKK1, FGF2, GALNT5, HAS3, HRAS, 1L18, NRG1, PTGER4 and VEGFC.

Specific genes involved in (8) concentration of lipid include at least one selected from the group consisting of ABCC3, ACPP, ACSL4, ADM, ALDH1A1, AMH, ANGPTL4, ATF3, C3, CCDC80, CD14, CEL, CERS6, CFB, CLN3, CLU, COL18A1, CPE, CTSS, CX3CL1, CXCL8, CYP 11A1, CYP27B1, DHRS3, D102, EDIL3, ELOV3, EPHX2, FAAH, FMO3, FOXA2, GATA6, GBA, GRK5, HRAS, IL18, IL18BP, LPL, LTC4S, MIF, NEILL NMU, NTRK2, NTS, NXPH4, PDE10A, PDK2, PLA2G4A, PLN2., PLPP3, PMCH, POR, PPARGCIA, PPP1R3C, PSEN2, PTGER4, PTGS2, RGCC, RILP, RRAD, S100A8, S100A9, SLC23A2, SLCO1B3, SNCA, SRC, TEN, TH, TSC1, UCN, UCP2 and UGT8.

Specific genes involved in (9) cell survival include at least one selected from the group consisting of ABCC3, ADGRE2, AFAPIL2, ALCAM, AMH, APLN, ASS1, ATF3, ATG12, BCL6, BMP2, BMP4, C3, CA9, CADM1, CALB1, CBLB, CCL5, CD82, CDK1, CEL, CHP2, CLN3, CLU, COL17A1, COL18A1, CTSB, CX3CL1, CXCL8, DAB2, DKK1, DUSP5, EPOR, ERBB3,FGF2, FGFR3, FN1, GADD45A, GATA6, GRB10, HK2, HNRNPUL2, HRAS, IGFBP2, IL18, JAG1, KIFC2, KLF4, KLF5, KLK6, KSR1, L1CAM, LIMS1, LRIG1, MAGED1, MCAM, MFAP5, MICAL2, MIF, MMP1, MMP13, MVP. NCSTN, NEIL1, NFIL3, NOV, NR4A2, NRG1, NTRK2, NUPR1, OPN3, PAK1, PCDHGC3, PDK1, PFDN4, PGF, PLA2G4A, POR, PPARGC1A, PPP1CB, PPP2R2A, PRPF8, PSEN2, PTGS2, PTN, PTPN13, RNF4, RORC, S100A8, S100A9, SERPINB2, SERPINF1, SFR1, SLC6A8, SLCO1B3, SNAI1, SNAI2, SNCA, SOCS2, SOX2, SPARC, SPRY1, SRC, SYK, TCF7L1, TFPI, TP73, TSC1, TUBGCP6, UCP2, VBP1, VCAN and VEGFC.

Specific genes involved in (10) chemotaxis of cells include at least one selected from the group consisting of APLN, BMP2, BMP4, C3, CCDC88A, CCL5, CLU, CSK, CX3CL1., CXCL6, CXCL8, ERBB3, FGF2, FN1, GRN, HDAC6, HRAS, IL18, KLRC4-KLRK1/KLRK1, LOX, LTB4R2, MIF, MYH10, MYLK, NOV, NRG1, PAK1, PGF, PTGS2, PTN, RTN4, S100A8, S100A9, SEMA3B, SEMA3D, SEMA3F, SERPINA1, SIRPA, SNAI2, SRC, SYK, TIRAP, TREM1, TSC1 and VEGFC.

Specific genes involved in (11) cell viability include at least one selected from the group consisting of ABCC3, ADGRE2, AFAPIL2, AME, APLN, ASS1, ATF3, ATG12, BCL6, BMP2, BMP4, C3, CA9, CADM1, CALB1, CCL5, CD82, CDK1, CEL, CHP2, CLN3, CLU, COL17A1, CTSB, CX3CL1, CXCL8, DAB2, DKK1, DUSP5, EPOR, ERBB3, FGF2, FGFR3, FN1, GADD45A, GATA6, HK2, HNRNPUL2, HRAS, IGFBP2, IL18, JAG1, KIFC2, KLF4, KLF5, KLK6, KSR1, LICAM, LRIG1, MCAM, MFAP5, MICAL2, MIF, MMP1, MMP13, MVP, NCSTN, NEIL1, NFIL3, NOV, NR4A2, NRG1, NIRK2, NUPR1, PAK1, PCDHGC3, PDK1, PFDN4, PGF, PLA2G4A, POR, PPARGC1A, PPP1CB, PPP2R2A, PRPF8, PSEN2, PTGS2, PTN, PTPN13, RNF4, RORC, S100A8, S100A9, SERPINB2, SERPINF1, SFR1, SLC6A8, SLCO1B3, SNAI1, SNAI2, SNCA, SOCS2, SOX2, SPARC, SPRY1, SRC, SYK, TCF7L1, TFPI, TP73, TSC1, TUBGCP6, VB1, VCAN and VEGFC.,

Specific genes involved in (12) permeability of vascular tissue include at least one selected from the group consisting of IL18, IL18BP MIF, NTS, PAK1 and VEGFC.

Specific genes involved in (13) chemotaxis include at least one selected from the group consisting of AMOT, APLN, BMP2, BMP4, C3, CCDC88A, CCL5, CLU, CSK, CX3CL1, CXCL6, CXCL8, ERBB3, FGF2, FN1, GRN, HDAC6, HRAS, IL18, KLRC4-KLRK1/KLRK1, L1CAM, LOX, LTB4R2, MIF, MYH10, MYLK, NOV, NRG1, PAK1, PGF, PIK3C2G, PTGS2, RTN4, S100A8, S100A9, SEMA3B, SEMA3D, SEMA3F, SERPINA1, SIRPA, SNAI2, SRC, SYK, TIRAP, TREM1, TSC1 and VEGFC.

Specific genes involved in (14) release of eicosanoid include at least one selected from the group consisting of BCL6, C3, CCL5, CTSB, CX3CL1, CXCL8, IL32, MIF, NMU, NRG1, NTS, PLA2G4A, PTGS2, SRC, SYK, T1 and CAM2.

Specific genes involved in (15) metabolism of carbohydrate include at least one selected from the group consisting of AGPAT1, ALDH1A1, ALDOC, APLN, BMP2, CBR3, CCL5, CHST15, CP, CS, CSGALNACT1, CTSB, CX3CL1, CXCL8, DIO2, DKK1, DPAGT1, FGF2, FOXA2, FUT2, GALNT5, GALT, GFPT2, GOT1, GRB10, GRKS, HAS3HK2, HRAS, IL18, LPL, MIF NR4A2, NRG1, NTS, PFKFB3, PPARGC1A, PPIP5K1, PPPICB, PPP1R3C, PIGER4, RGS2, SIAE, SLC23A2, SLCSA3, SNCA, ST6GAL1, ST6GALNAC1, SYK, TIRAP, UGT1A6 and VEGFC.

Specific genes involved in (16) release of fatty acid include at least one selected from the group consisting of BCL6, C3, CCL5, CTSB, CX3CL1, CXCL8, IL32, MIF, NMU, NRG1, NTS, PLA2G4A, PPARGC1A, PTGS2, SRC, SYK, TICAM2 and UCN.

Specific genes involved in (17) synthesis of DNA include at least one selected from the group consisting of ADM, AFAP1L2ALDH3A1, AMH, ATF3, BMP2, BMP4, BMPRIB, CCDC88A, CCL5, CLU, CXCL8, ERBB3, FGF2, FN1, GRN, HRAS, IGFBP2, IGFBP4, IGFBP6, KIAA0101., KSR1, MIF, NCOA2, NOV, NRG1, NTS, PGF, PTGS2, PTN, RGS12, RRAD, SERPINA1, SNAI1, SPARC, SRC and TP73.

Specific genes involved in (18) migration of tumor cell lines include at least one selected from the group consisting of ACSL4. ALCAM, ATF3, BMP2., CA9, CALML3, CCDC88A, CCL5, CD82, CHP2, CLU, COL18A1, CSK, CST6, CTSB, CX3CL1, CXCL8, DAB2, DDX58, DPAGT1, EPOR, ERBB3, F3, FGF2, FN1, FOXQ1, GRN, HAS3, HDAC6, HRAS, IGFBP2, IGFBP4, IGFBP6, IL32, ITGB6, KLF4, KLF5, KLK6, KLRC4-KLRK1/KLRK1, KRT8, L1CAM, LOX, MALAT1, MCAM, MIF, MMPI, MYH10, NOV, NR4A2, NRG1, NTRK2, PAK1, PDK1, PGF, PHLDA1, PTGER4, PTGS2, PTN, RAB21, RALGAPA2, S100A8, S100A9, SEMA3F, SERPINA1, SERPINF1, SIRPA, SNAI1, SNAI2, SPARC, SRC, ST6GAL1, SYK, TFPI, TP73, TSC1, VCAN, VEGFC and VSNL1.

Specific genes involved in (19) differentiation of tumor cell lines include at least one selected from the group consisting of AJUBA, AKRIC3, BCL6, BMP2, BMP4, CD14, COL18A1, DAB2, EPOR, FEZ1, FGF2, FN1, FST, GCNT1, HRAS, JAG1, KLF4, KMT2A, MIF, mir-25, NOV, NRG1, NTRK2, PTGER4, PTGS2, SERPINB2, SIRPA, SNCA, SOX2, SPARC, SRC, SSBP2, TP73 and VCAN.

Specific genes involved in (20) proliferation of tumor cells include at least one selected from the group consisting of AFAPIL2, ATF3, BMP2, BMP4, CA9, CD82, CST6, CTSB, CTSS, CTSV, CXCL8, DKK1, ERBB3, F3, FGF2, FGFR3, FST, GLDC, GRN, HDAC6, HRAS, IGFBP2, IL18, IL32, JAG1, KMT2A, LGALS7/LGALS7B, LOX, LUM, MCAM, MIF, mir-25, MK167, NOV, NR4A2, NRG1, NTS, PFKFB3, PTGS2, PTN, PTPN13, RORC, S100A9, SEMA3B, SNAI1, SNAI2, SOX2, SRC, TP73, TRIM22, VCAN and VEGFC.

Specific genes involved in (21) tyrosine phosphorylation include at least one selected from the group consisting of ADM, AFAPIL2, CADM1, CBLB, CCL5, CHST15, CNTN1, CSK, CX3CL1, EPOR, FGF2, FN1, MIF, MMPI, NRG1, PAG1, PSEN2, PTN, PTPN13, SRC, ST6GAL1, SYK and VEGFC.

Specific genes involved in (22) development of abdomen include at least one selected from the group consisting of ADM, ALDH1A1, AMH, APLN, ARID5B, ATF3, BMP2, BMP4, CADM1, CD14, CLU, CTSS, CX3CL1, CYP11A1, CYP27B DKK1, DYNC2LI1, EPOR, ERBB3, FGF2, FOXA2, FOXD1, FST, GATA6, GCNT1, HDAC6, HRAS, IGFBP2, JAG1, KMT2A, KRT8, LIN7C, NFIL3, NRG4, NTRK2, PGF, PTGS2, RDH10, RORC, SCHIP1, SPRY1, SRC, STK36, IF, TP73, TSC1 and VEGFC.

Specific genes involved in (23) metastasis of cells include at least one selected from the group consisting of AGR2, ALCAM, ANGPTL4, ATF3, BMP2, BMP4, CCDC88A, CD82, COL18A1, CTSB, CXCL6, CXCL8, DAPK1, F3, FGF2, FN1, IL18, LOX, LTB4R2, MALAT1, MBD2, MCAM, MMP1, MMP10, NBEAL2, NTRK2, PGF, PTGS2, SERPINA1, SNAI1, SNAI2, SRC and VEGFC.

Specific genes involved in (24) cell movement include at least one selected from the group consisting of ACSL4, ADARB1 ADM, AGR2, AJUBA, ALCAM, ALPP, AMOT, ANGPTL4, APLN, ARID5B, ATF3, ATGI2, ATP2B4, BCAS3, BMP2, BMP4, C3, CA9, CADM1, CADPS2, CALML3, CBLB, CCDC88A, CCL5, CD14, CD58, CD82, CDK1, CFB, CHP2, CLU, CNTN1, COL17A1, COL18A1, CSK, CST6, CTSB, CTSS, CTSV, CX3CL1, CXCL6, CXCL8, DAB2, DDX58, DIO2, DKK1, DNAH11, DNAJB4, DPAGT1, DSG3, ECSCR, EDIL3, EMD, EPOR, EPS8, ERBB3, F3, FAAH, FGF13, FGF2, FGFR3, FN1, FOXA2, FOXQ1, FST, GADD45A, GALNT1, GATA6, GBA, GCNT1, GJB2, GRN, HAS3, HDAC6, HEY1, HRAS, ICAM2, IGFBP2, IGFBP4, IGFBP6, IL18, IL18BP, IL32, ITGB6, JAG1, KCNK2, KCNK5, KLF4, KLF5, KLK6, KLRC4-KLRK1/KLR1, KMT2A, KRT10, KRT16, KRT8, LICAM, LIMS1, LOX, LRIG1, LTB4R2, LTC4S, LUM, LYPD3, MALAT1, MCAM, MESP1, MIF, mir-25, MMP1, MMP10, MMP13, MYH10, MYLK, NFIL3, NMU, NOV, NR4A2, NRG1, NTRK2, NTS, PAK1, PAPPA, PARD6B, PCOLCE2, PDK1, PGF, PHLDA1, PIK3C2G, PLA2G4A, PLPP3, PMCH, PTGER4, PTGS2, PTN, PTPRR, RAB21, RALGAPA2, RAP2B, RGCC, RGS16, RORC, RTN4, S100A2, S100A8, S100A, SCHIP1, SEMA3B, SEMA3D, SEMA3F, SEMA6D, SERPINA1, SERPINB2, SERPINE2, SERPINF1, SH3BP1, SIRPA, SNAH, SNAI1, SNAI2, SNCA, SOCS2, SOX2, SPARC, SRC, ST6GAL1, SYK, TFPI, TIRAP, TNS3, TP73, TREM1, TSC1, TUBA1A, UCN, UCP2, UTRN, VCAN, VEGFC, VSNL1, WISP2 and WLS.

Specific genes involved in (25) movement of vascular endothelial cells include at least one selected from the group consisting of ADM, BMP4, COL18A1, CXCL8, DIO2, DKK1, ECSCR, FGF13, FGF2, FN1, GATA6, KMT2A, LOX, NRG1, PLA2G4A, PTGS2, PTN, RTN4, SEMA3D, SNAI2, SPARC, SRC and VEGFC.

Specific genes involved in (26) progression of tumor include at least one selected from the group consisting of AGR2, ALCAM, CA9, CCL5, CLU, CXCL8, ERBB3, F3, FGF2, IGFBP2, 1IGFBP4, IGFBP6, IL18, MCAM, MIF, PAK1, PTGS2, SERPINF1, SOX2, SRC and TSC1.

Specific genes involved in (27) organization of cytoskeleton include at least one selected from the group consisting of ABLIM3, ADM, AJUBA, AKAP2, AMOT, ANGPTL4, ATF3, BCAS3, BCL6, BICDL1, BMP2, BMP4, C2CD3, C3, CALML3, CAPN6, CCDC88A, CCL5, CD82, CDK1, CGN, CLN3, CLU, CNTN1, CSK, CTSS, CTSV, CUL7, CXCL8, DAPK1, DYNC2LI1, EPHX2, EPS8, F3, FEZ1, FGF13, FGF2, FHL3, FN1, GAS2L3, GRK5, GRN, HDAC6, HERC1, HRAS, IFT122, IGSF9, KLF5, KRT16, KRT4, L1CAM, LAMB2, LOX, LRRC4C, MAEA, MAOA, MICAL2, MNS1, MYH10, NRG1, NTRK2, NTS, PAK1, PARD6B, PKP1, PLXNA3, PSEN2, PTGS2, PTN, PVRL1, RAB21, RGS2, BRAD, RIN4, S100A8, S100A9, SEMA3D, SEMA3F, SERPINF1, SIPA1L1, SIRPA, SLITRK6, SNCA, SNX10, SPARC, SRC, STK36, SYK, TF, TSC1, TUBGCP6, UCN, UGT8, VEGFC and WISP2.

Specific genes involved in (29) activation of cells include at least one selected from the group consisting of APLN, ATF3, BLNK, BMP2, C3, CADM1, CBLB, CCL5, CD14, CD58, CPE, CSK, CTSS, CX3CL1, CXCL6, CXCL8, DDX58, DKK1, ENTPD2, F3, FGF2, FGFR3, FN1FOXA2, FOXD1, FZD5, GADD45A, GRN, HAS3, HDAC6, HLA-DMA, HLA-DPA1, HNRNPD, ICA1, ICAM2, IL18, IL32, ITGB6, KLF4, KLRC1, KLRC2, KLRC4-KLRK1/KLRK1, KRT8, KSR1, LIN7C, MIF, MMP1, MMP13, NBEAL2, NFIL3, NMU, NOV, NRG1, NTS, PAG1, PLA2G4A, PSEN2, PTGER4, PTGS2, RAP2B, RGCC, RORC, S100A8, S100A9, SATB1, SERPINA1, SERPINE2, SERPINF1, SERPINF2, SIAE, SIRPA, SNCA SOCS2, SRC, ST6GAL1, SYK, TF, TIRAP, TP73, TREM1 and VCAN.

In the present invention, as to increased or decreased expression of genes related to the above-mentioned functions, in the case of a gene related to enhancement of the function, the expression level is 2 times or more, preferably 4 to 10 times, more preferably 7 to 10 times than the expression level of each gene in a cancer non-stem cell (a cancer cell that is not a stem cell) from the same separation source, or in the case of a gene related to suppression of the function, the expression level is ½ times or less, preferably ¼ to 1/10 times, more preferably 1/7 to 1/10 times than the expression level of each gene in a cancer non-stem cell (a cancer cell that is not a stem cell) from the same separation source.

The expression level of each gene related to the function can be determined by a known gene analysis method using a next-generation sequencer or the like.

The origin of the cancer stem cells of the present invention is not particularly limited, but depending on the purpose of use of the cancer stem cells etc., they are appropriately selected from those derived from mammals such as human, mouse, rat, hamster, rabbit, cat, dog, sheep, pig, cow, goat and monkey. When prepared cancer stem cells are used as a drug discovery tool for human cancer or a tool for developing a diagnostic agent, it is preferable that the cancer stem cells be derived from human.

Specific examples of the deposited line that facilitates acquisition of the cancer stem cells of the present invention include Panc-1 3-4 CST 001 line. The Panc-1 3-4 CST 001 line has been internationally deposited under Accession No. NITE BP-02449 at Patent Microorganisms Depositary, National Institute of Technology and Evaluation (2-5-8 Kazusakamatari, Kisarazu-shi, Chiba, Japan). The date of receipt of the domestic deposit in Japan is on Mar. 22, 2017, and the date of transfer to the international deposit under the Budapest Treaty is on Mar. 20, 2018, in the Panc-1 3-4 CST 001 line, human-derived cancer stem cells (ZsGreen+/CD44v9^{high} cells) are included, even a single cell of Which can form tumor tissue, highly express CD44v9, and have increased expression level of genes related to the functions (1) to (29).

2. Method for Establishing Cancer Stem Cell Line

The cancer stem cells of the present invention can be obtained by separating and enriching cells having a low metabolic function of 26s proteosome from a cancer cell group containing cancer stem cells.

The “cancer cell group containing cancer stem cells” may be a cell group obtained from a cancer tissue removed from a cancer patient, or may be a cell group stocked as cancer cells. Usually, in the cell group stocked as a cancer tissue or cancer cells, cancer stem cells are contained in a very small proportion.

The cancer type of the cancer cell group is not particularly limited, but any cancer cell group derived from any cancer can be used. Specific examples of the origin of the cancer cell group used in the present invention include pancreatic cancer, colorectal cancer, large bowel cancer, esophageal cancer, gastric cancer, liver cancer, cholangiocarcinoma, lung cancer and skin cancer. Among them, pancreatic cancer is preferable.

The origin of the cancer cell group is not particularly limited, but any group derived from any mammal origin can be used, and the specific examples or preferable examples etc. are as described in the column of “1. Characteristics of cancer stem cell etc.”.

In the present invention, “cells having a low metabolic function of 26s proteosome” are cells having a reduced metabolic function of 26s proteosome and having properties as cancer stem cells, The “cells having a low metabolic function of 26s proteosome” may be, for example, cells on which a labeled substance is detected after the labeled ornithine decarboxylase-degron as described below is transduced, followed by culture and selection for 2 to 3 weeks.

There is no particular limitation on a method for separating and enriching cells having a low metabolic function of 26s proteosome from a cancer cell group, but, for example, a method for labeling cells having a low metabolic function of 26s proteosome and enriching the labeled cells is included.

There is no particular limitation on a method for labeling cells having a low metabolic function of 26s proteosome contained in a cancer cell group, but a method for transducing a labeled omithine decarboxylase-degron (ODC-Degron) into a cancer cell group is preferably included. Because cells having a low metabolic function of 26s proteosome cannot degrade the ODC-Degron, a label introduced to the ODC-Degron can sufficiently accumulate in the cells to be labeled.

The type of a labeling substance used for labeling the ODC-Degron is not particularly limited, but examples thereof include fluorescent proteins such as GFP, YFP ZsYellow, Dsred, mCherry, mOrange and mCerulean. In addition, transduction of the labeled ODC-Degron into a cancer cell group can be performed by a known method such as a method including using a vector; calcium phosphate method; lipofection method; electroporation method; or microinjection method.

In a cancer cell group, cells having a low metabolic function of 26s proteosome exist only in a very small proportion. For this reason, labeled cells having a low metabolic function of 26s proteosome are enriched.

The method for enriching labeled cells having a low metabolic function of 26s proteosome is not particularly limited, but from the viewpoint of efficiently obtaining cancer stem cells having high tumorigenicity, a method for fractionating cells having high detected amount of labeling substance by FACS or the like is preferably included.

In addition, enrichment of cells having a. low metabolic function of 26s proteosome may be performed so as to contain 60% or more, preferably 70% or more, more preferably 75% or more, particularly preferably 80% or more cells having a low metabolic function of 26s proteosome with respect to the total number of enriched cells.

The enriched cells having a low metabolic function of 26s proteosome in this way have so excellent tumorigenicity as cancer stem cells that tumor can be formed even with as low as 200 or less cells.

In addition, from the cancer stem cells (cells having a low metabolic function of 26s proteosome) obtained in this way, further separating cancer stein cells highly expressing CD44v9 can provide cancer stem cells having remarkably high tumorigenicity.

Separating cancer stem cells highly expressing CD44v9 can be performed using a known method such as cell sorting by FACS analysis using an anti-CD44v9 antibody. In addition, indicators of “highly expressing CD44v9” are as described in the column of “1. Characteristics of cancer stem cell etc.”.

3. Use of Cancer Stem Cell

The cancer stem cells of the present invention can be used to elucidate mechanisms of cancer development, progression, metastasis and the like by analyzing their characteristics, and further allows for screening of substances capable of inducing their differentiation or controlling proliferation. Specifically, by bringing test substances (candidate substances to be subjected to screening) into contact with the above-obtained cancer stem cells, measuring the presence or absence of differentiation of the cancer stem cells, and selecting test substances that differentiate the cancer stem cells, it is possible to screen substances that induce cancer stem cells to differentiate. In addition, by bringing test substances (candidate substances to be subjected to screening) into contact with the above-obtained cancer stem cells, measuring the degree of proliferation of the cancer stem cells, and selecting test substances that suppress proliferation of the cancer stem cells or test substances that promote proliferation of the cancer stem cells, it is possible to screen substances that can suppress proliferation of cancer stem cells or substances that promote proliferation of cancer stem cells.

In addition, because cancer stem cells are responsible for development, progression and metastasis of cancer, the cancer stem cells of the present invention can also be used for screening of a cancer therapeutic agent, evaluation of efficacy of a cancer therapeutic agent, and the like. Specifically, by bringing test substances (candidate substances to be subjected to screening) into contact with the cancer stem cells of the present invention, measuring the presence or absence of growth of the cancer stem cells, and selecting test substances that inhibit growth of the cancer stem cells, it is possible to screen a cancer therapeutic agent. In addition, by bringing a cancer therapeutic agent into contact with the cancer stem cells of the present invention, and measuring the degree of growth inhibition of the cancer stem cells, it can be evaluated that the greater the degree of growth inhibition of cancer stem cells, the more effective the cancer therapeutic agent, or the smaller the degree of growth inhibition of cancer stem cells, the lower the efficacy of the cancer therapeutic agent. Note that the “growth inhibition of cancer stem cells” involves not only a case where the growth of cancer stem cells is stopped or the growth ability thereof is reduced, but also a case where cancer stem cells are killed.

### EXAMPLES

Hereinafter, a description is made of the present invention with reference to Examples. However, the present invention is not to be construed as being limited to the following Examples.

### Example 1

### Separation and Characteristic Analysis of Cancer Stem Cell

1. Separation of Cancer Stem Cell

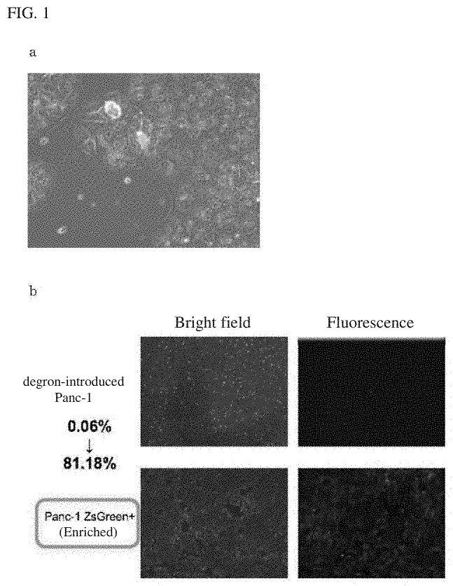

A pancreatic cancer cell line (Pane-1) was transduced with a GFP-fluorescently labeled ODC (Ornithine Decarboxylase)-Degron system using a retrovirus vector. The base sequence coding the used ODC-Degron is as set forth in SEQ ID NO: 1. After transduction, culture and selection were performed for 2 to 3 weeks, and the cells were observed. The results are shown in FIG. 1a. As can be seen from FIG. 1a, even when the ODC-degron system was introduced into the pancreatic cancer cell line Panc-1, only about 0:06% cells (cancer stem cells) emitting fluorescence were obtained in total. Such a small number of cancer stem cells do not allow for a series of experiments including drug screening.

For this reason, next, cells (cancer stem cells) emitting fluorescence (green) were sorted using a cell sorter (SH800Z purity sorting mode by SONY Corporation), followed by 2 weeks of culture in a 10 cm dish containing 10 ml DMEM (10% FBS, 1 mg/ml of G418) for enrichment of the fluorescent cells (cancer stem cells). The results of the observed enriched cells are shown in FIG. 1b. In this way, enrichment of the fluorescent cells (cancer stem cells) provided a cancer stem cell population in which cells having observed fluorescent coloring (i.e., cancer stem cells) accounted for about 81,18% in total. The obtained cancer stem cell population was designated as ZsGreen+.

2. Characteristic Analysis of Cancer Stem Cell

As to the obtained cancer stem cell population (ZsGreen+), in order to confirm the characteristics of the cancer stem cells, the ability to form spheres, anticancer drug resistance, ability to express a stem cell marker, and asymmetric division potency were confirmed. For comparison, a non-cancerous stem cell population (ZsGreen−) that was obtained through introduction of the ODC-degron system into the pancreatic cancer cell line Paric-1 and did not emit fluorescence was also subjected to characteristic analysis in the same way.

As to the ability to form spheres, using 96-well Ultra Low Cluster Plate, each cell population was seeded at 100 to 3000 cells/well and cultured at 37° C. in (DMEM) medium, and then the state of cell was observed over 2 weeks for confirmation,

As to the anticancer drug resistance, using a 96-well plate, each cell population was seeded at 5000 to 10000 cells/well and cultured at 37° C. for 2 to 4 days in a medium (DMEM medium containing 10% by volume FBS) supplemented with 2 or 5 μM oxaliplatin (L-OHP), and then the number of living cells were counted to determine the cell viability (%).

As to the expression of stem cell marker, expression of stem cell markers Bmil and CD44v9, and a cancer stem cell-specific marker Dclk1 was measured by qPCR and Western blotting. The expression levels of Bmil and Dclk1 were corrected with the expression level of the housekeeping gene (GAPDH), and the expression level of CD44v9 was corrected with the expression level of the housekeeping gene (Actin).

As to the asymmetric division potency, using ibidi 35 mm Plate, each cell population was seeded at 1000 to 10000 cells/plate and cultured at 37° C. for 1 day in a medium (DMEM medium containing 10% by volume FBS), and then the condition of cells was continuously observed over 1 week for confirmation. Note that cancer stein cells have the property of asymmetrically dividing to produce daughter cells having different properties, resulting in genetically heterogeneous cell division.

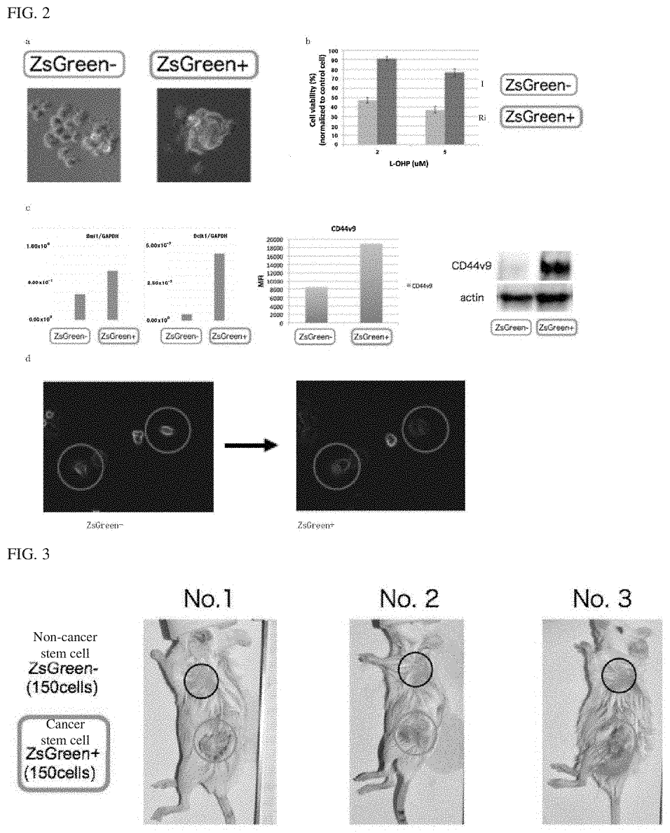

In FIG. 2, the result of confirmation of the presence or absence of characteristics of cancer stem cells is shown for the cancer stem cell population (ZsGreen+) and the non-cancer stem cell population (ZsGreen−, negative control). In FIG. 2, the result of evaluation of the ability to form spheres is shown in a, the result of evaluation of the anticancer drug resistance in b, the result of evaluation of the ability to express a stem cell marker in c, and the result of evaluation of the asymmetric division potency in d. As a result, the cancer stem cell population (ZsGreen+) has an ability to form spheres, anticancer drug resistance and asymmetric division potency, and observed expression of the stem cell markers and the cancer stein cell-specific marker, confirming that the population has characteristics of cancer stem cells.

3. Analysis of Tumorigenicity

Following tests were performed to confirm the tumorigenicity of the obtained cancer stem cell population (ZsGreen+). First, subcutaneous transplantation of the cancer stem cell population (ZsGreen+) was performed to 6 to 8 weeks old. SCID beige mice (n=3) so as to provide 150 cells each. Six weeks later, the site where the cells were transplanted was observed to confirm the presence or absence of tumor formation. For comparison, also for the non-cancer stem cell population (ZsGreen−), the tumorigenicity was analyzed in the same way.

The obtained result is shown in FIG. 3. As a result, the transplantation of only 150 cells from the cancer stem cell population (ZsGreen+) successfully formed a tumor having a diameter of about 10 mm or more, making it clear that the tumorigenicity is high. On the other hand, the transplantation of 150 cells from the non-cancer stem cell population (ZsGreen−) failed to form a tumor.

### Example 2

### Separation and Characteristic Analysis of Cancer Stem Cell Highly Expressing CD44v9

1. Separation of Cancer Stem Cell Highly Expressing CD44v9

As to the cancer stem cell population (ZsGreen+) and the non-cancer stem cell population (ZsGreen−) obtained in Example 1, they were divided into a group highly expressing CD44v9 or a group not highly expressing CD44v9 depending on the expression level of CD44v9 by FACS using an anti-CD44v9 antibody. Compared to cells having the lowest expression level of CD44v9 (detection threshold) in the cell population, cells having 10 times or ore expression level of CD44v9 were grouped as high expression of CD44v9, and cells having less than 5 times expression level of CD44v9 were as non-high expression of CD44v9. Hereinafter, a cell population having high expression of CD44v9 is denoted as “ZsGreen+/CD44v9^{high}” in the cancer stem cell population (ZsGreen+), a cell population non-highly expressing CD44v9 in the cancer stem cell population (ZsGreen+) is as “ZsGreen±/CD44v9⁻”, a cell population having high expression of CD44v9 in the non-cancer stem cell population (ZsGreen−) is as “ZsGreen−/CD44v9^{high}”, and a cell population non-highly expressing CD44v9 in the non-cancer stem cell population (ZsGreen−) is as “ZsGreen−/CD44v9⁻”.

2. Analysis of Tumorigenicity

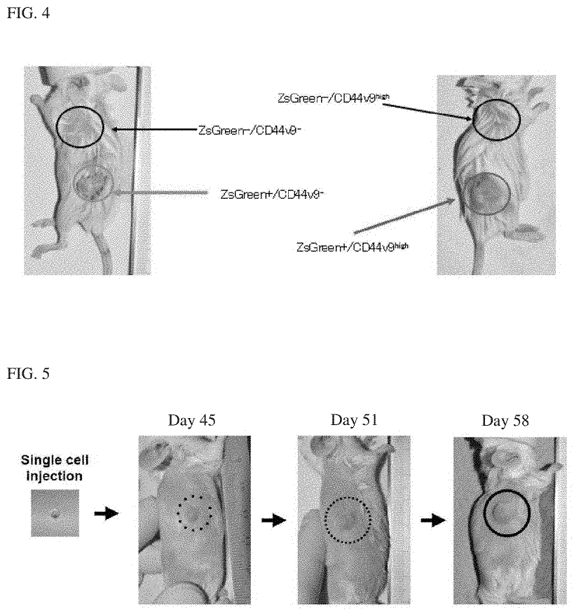

Under the same condition as that of “3. Analysis of tumorigenicity” described in Example 1, the tumorigenicity was analyzed. The obtained result is shown in FIG. 4. Six weeks after transplantation, for the ZsGreen−/CD44v9^{high} cell population, no tumorigenesis was observed via transplantation of 150 cells, whereas for both of the ZsGreen+/CD44v9^{high} and ZsGreen+/CD44v9 cell populations, formation of a tumor having a diameter of about 15 mm was observed via transplantation of 150 cells.

3. Gene Analysis By Next-Generation Sequencing

Intermolecular network pathway analysis was used to analyze genetic differences among the ZsGreen−/CD44v9^{high}, ZsGreen+/CD44v9⁻ and ZsGreen−/CD44v9⁻ cell populations.

Specifically, using TruSeq stranded mRNA sample prep kit (Illumina, San Diego, Calif.), an mRNA library was prepared according to the manufacturer's instruction. Sequencing was performed using Illumina HiSeq 2500 platform (75-base single-end mode). Base calling was performed using Illumina Casava 1.8.2 software. The resultant sequences were mapped with respect to the mouse reference genome sequence (mm10) using TopHat v2.0.13 in combination with Bowne2 ver. 2.2.3 and SAMtools ver. 0.1.19TopHat v2.0.13. One million read sequences were mapped, and the normalized gene expression level when the length of the transcript (fragment) was 1 kilobase was calculated using Cuffnorm version 2.2,1.

Next, principal component analysis was performed using omics analysis software Subio platform manufactured by Subio Inc. (ver. 1.19; Subio, Kagoshima, Japan), and pathway analysis was performed using software Ingenuity Pathway Analysis (IPA, QIAGEN Redwood City),

Compared to the ZsGreen−/CD44v9⁻ cell population, about 600 gene groups having a fold change of ±2, p<0.05 were observed in the ZsGreen−/CD449⁻ cell population. On the other hand, compared to the ZsGreen-/CD44v9⁻ cell population, about 760 gene groups having a fold change of ±2, p<0.05 were observed in the ZsGreen+/CD44v9^{high} cell population. When IPA analysis was performed on the ZsGreen+/CD44v9⁻ and ZsGreen+/CD44v9^{high} cell populations, a clear difference was observed between them, as shown in Table 1. in other words, compared to the ZsGreen+/CD44v9⁻ cell population (vs ZsGreen−/CD44v9⁻), in the ZsGreend+/CD44v9^{high} cell population (vs ZsGreen−/CD44v9⁻), activation (activation z-score was set to 2.0 or more) of pathways involved in growth/progression of tumor tissue and metastasis/differentiation/proliferation of tumor cells such as growth of tumor, migration of tumor cell lines, differentiation of tumor cell lines, proliferation of tumor cells and progression of tumor was recognized. On the other hand, in the ZsGreen+/CD44v9⁻cell population (vs ZsGreen−/CD44v9⁻), there were no tumor-related activated pathways. From the above results, it has been found that the ZsGreen+/CD44v9^{high} cell population is a group of extremely active tumor cells.

TABLE 1

| | | | |
|---|---|---|---|
| | | | Activation |
| Diseases or Functions Annotation | p-Value | Predicted | z-score |

ZsGreen+/CD44v9⁻ CELL POPULATION (VS ZsGreen−/CD44v9⁻)

| | | | |
|---|---|---|---|
| Organ Degeneration | 0.000147 | Increased | 2.946 |
| hepalic steatosis | 0.000479 | Increased | 2.681 |
| size of lesion | 0.00114 | Increased | 2.452 |
| accumulation of lipid | 0.00068 | Increased | 2.448 |
| Growth Failure | 0.000221 | Increased | 2.395 |
| signaling of cells | 0.000472 | Increased | 2.213 |
| morbidity or mortality | 7.51E−07 | Increased | 2.21 |
| organismal death | 5.96E−07 | Increased | 2.159 |
| concentration of phospholipid | 0.000252 | Increased | 2.102 |
| mitosis of breast cancer cell lines | 0.000817 | Increased | 2 |

ZsGreen+/CD44v9^{high} CELL POPULATION (VS ZsGreen−/CD44v9⁻)

| | | | |
|---|---|---|---|
| growth of tumor | 8.53E−12 | Increased | 3.178 |
| cell movement of endothelial | 0.000084 | Increased | 3.054 |
| cell lines | | | |
| migration of endothelial cell lines | 3.75E−05 | Increased | 2.917 |
| synthesis of carbohydrate | 0.000114 | Increased | 2.737 |
| synthesis of polysaccharide | 4.17E−05 | Increased | 2.56 |
| phosphorylation of protein | 1.01E−06 | Increased | 2.551 |
| synthesis of glycosaminoglycan | 4.42E−06 | Increased | 2.55 |
| Concentration of lipid | 9.78E−09 | Increased | 2.516 |
| Cell survival | 6.17E−10 | Increased | 2.494 |
| chemotaxis of cells | 8.91E−06 | Increased | 2.432 |
| cell viability | 3.89E−09 | Increased | 2.423 |
| permeability of vascular tissue | 5.45E−05 | Increased | 2.401 |
| chemotaxis | 1.79E−06 | Increased | 2.399 |
| release of eicosanoid | 0.000036 | Increased | 2.347 |
| metabolism of carbohydrate | 4.29E−06 | Increased | 2.306 |
| release of fatty acid | 0.000043 | Increased | 2.259 |
| synthesis of DNA | 8.87E−06 | Increased | 2.258 |
| migration of tumor cell lines | 3.86E−13 | Increased | 2.249 |
| Organ Degeneration | 6.77E−06 | increased | 2.229 |
| differentiation of tumor cell lines | 1.83E−05 | Increased | 2.195 |
| proliferation of tumor cells | 1.56E−10 | Increased | 2.178 |
| tyrosine phosphorylation | 6.22E−07 | Increased | 2.17 |
| development of abdomen | 2.65E−05 | Increased | 2.133 |
| metastasis of cells | 2.16E−09 | Increased | 2.132 |
| cell movement | 3.4E−17 | Increased | 2.12 |
| movement of vascular endothelial | 3.46E−07 | Increased | 2.087 |
| cells | | | |
| progression of tumor | 1.32E−07 | Increased | 2.068 |
| organization of cytoskeleton | 5.89E−05 | Increased | 2.058 |
| activation of cells | 3.95E−08 | Increased | 2.047 |
| migration of breast cancer cell lines | 0.000169 | Increased | 2.021 |

### Example 3

Establishment of cancer stem cell line from ZsGreen+/CD44v9^{high} cell population and analysis of tumorigenicity thereof.

1. Establishment of Cancer Stem Cell Line

The ZsGreen+/CD44v9^{high} cell population was cultured in a medium containing an antibiotic G418, and then mouse-derived cells were removed for cell line establishment. The established cancer stem cell line was subjected to pure culture, and the resultant cells were stocked (1×10⁶ cells/ml of CELLBANKER cell cryopreservation solution). One of the established cancer stem cell lines was designated as “Panc-1 3-4 CST 001 line” and deposited at National Institute of Technology and. Evaluation (Accession Number: NITE BP-02449).

2. Analysis of Tumorigenicity

The tumorigenicity of the established cancer stem cell lines was analyzed. For the test method, the same condition as that of “3. Analysis of tumorigenicity” described in Example 1 was adopted, except that the number of cells to be administered was one and the cell was transplanted in an embedded state in a base (DMEM: matrigel (volume ratio)=1:1, 100 μl in total).

The obtained result is shown in FIG. 5. As a result, even when only one cell from the established cancer stem cell line was transplanted, tumor formation was observed on the day 45th after transplantation, and drastic increase in tumor tissue was observed over the next two weeks.

1. A cancer stem cell, which is capable of forming tumor tissue with 200 or less cells in vivo.

2. The cancer stem cell according to claim 1, which is capable of forming tumor tissue with one cell in vivo.

3. The cancer stem cell according to claim 1, which highly expresses CD44v9.

4. The cancer stem cell according to, which is Panc-1 3-4 CST 001 line (Accession number: NITE BP-02449).

5. A method for separating a cancer stem cell, comprising a step of separating and enriching a cell having a low metabolic function of 26s proteosome from a cancer cell group containing the cancer stem cell.

6. The method for separating a cancer stem cell according to claim 5, wherein the step of separating and enriching a cell having a low metabolic function of 26s proteosome is conducted by transducing a labeled ornithine decarboxylase-degron into the cancer cell group.

7. The method for separating a cancer stem cell according to claim 5, comprising further separating a cell highly expressing CD44v9 from the cell having a low metabolic function of 26s proteosome, the cell being enriched.

8. A method for screening a cancer therapeutic agent, comprising screening a cancer therapeutic agent using the cancer stem cell according to claim 1.

9. A method for evaluating efficacy of a cancer therapeutic agent, comprising evaluating efficacy of a cancer therapeutic agent using the cancer stem cell according to claim 1.

10. The cancer stem cell according to claim 2, which highly expresses CD44v9.

11. The method for separating a cancer stem cell according to claim 6, comprising further separating a cell highly expressing CD44v9 from the cell having a low metabolic function of 26s proteosome, the cell being enriched.


SEQUENCE LISTING (1 sequence)

SEQ ID NO: 1 - DNA, 858 bp, Homo sapiens
atggcccagtccaagcacggcctgaccaaggagatgaccatgaagtaccgcatggagggctgcgtggatggccacaagttcgtgatcaccggcgagggcatcggctaccccttcaagggcaagcaggccatcaacctgtgcgtggtggagggcggccccttgcccttcgccgaggacatcttgtccgccgccttcaactacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgcccccgcggtcacggccaatgtggcaactcatgaaacagatccagagccatggcttcccgccggaggtggaggagcaggatgatggcacgctgcccatgtcttgtgcccaggagagcgggatggaccgtcaccctgcagcctgtgcttctgctaggatcaatgtgtag